(19) United States
(12) Patent Application Publication
(10) Pub. No.: US 2022/0176053 A1
(43) Pub. Date: Jun. 9, 2022

(54) DEVICE FOR HEATING ENDOTRACHEAL TUBES PREPARATORY TO NASAL INTUBATION

(71) Applicant: Matthew Arnall, Mesa, AZ (US)

(72) Inventor: Matthew Arnall, Mesa, AZ (US)

(21) Appl. No.: 17/116,951

(22) Filed: Dec. 9, 2020

(51) Int. Cl. A61M 16/04

(52) U.S. Cl. CPC A61M 16/0402; A61M 16/0461; A61M 16/0463; A61M 2205/502; A61M 2205/3368; A61M 2205/3633; A61M 2205/02; A61M 16/047

(57) ABSTRACT

A heating device for heating surgical tubes is disclosed herein. The heating device comprises a hollow housing configured to receive the surgical tubes therein. A heating element provided on the hollow housing. A first sensor is assembled on the heating element and configured to sense the temperature of the heating element. A controller is coupled to the heating element and the first sensor, wherein the controller is configured to receive an input associated with a required heating temperature from a user for heating the surgical tubes, and accordingly regulate the operation of the heating element based on the required heating temperature and an actual temperature sensed via the first sensor.

### TECHNICAL FIELD

The present disclosure relates generally to heating endotracheal tubes preparatory to nasal intubation.

### BACKGROUND

Tracheal intubation is challenging and requires skill and experience that can only be developed over years of practice. There are many instances where intubation from the nose is preferred over intubation through mouth. However, intubation via nose is a more challenging and difficult route of intubation.

Most endotracheal tubes are made of plastics that are flexible across the length of the tube but fairly rigid on the leading edge of the tube. When the tube is passed over the nasal cavity, the rigidity of the plastic often tends to traumatize the delicate tissues inside the floor of the nasal cavity and the turbinates. The delicate tissues in the nasal cavity are prone to heavy bleeding when traumatized. This is not desired because bleeding hampers the visibility of the practitioner during the course of the procedure. Furthermore, the traumatized tissue may start bleeding again when the tube is removed, which may cause postoperative airway complications from bleeding into the airway after the procedure. In addition, in case of oral intubation, the practitioner has ample play to maneuver the tube laterally or vertically to align and enter the tube in the trachea. Conversely, performing intubation nasally and aligning the tube with the trachea may be a bit more difficult due to constricted, narrow, and ridged profile of the nasal cavity. Even if such a nasal intubation is successful, damage to the delicate nasal tissues may not be avoided, which may cause discomfort to the patient after the procedure.

### SUMMARY

A device for heating surgical tubes preparatory to nasal intubation is disclosed herein. The device comprises a hollow housing configured to receive the surgical tubes therein. A heating element is provided on the hollow housing. A first sensor is assembled adjacent the heating element, and configured to sense the temperature of the heating element. A controller is coupled to the heating element and the first sensor, wherein the controller is configured to receive an input associated with a required heating temperature from a user for heating the surgical tubes, and accordingly regulate the operation of the heating element based on the required heating temperature and an actual temperature sensed via the first sensor.

In another non-limiting exemplary embodiment, the heating element is a heating pad.

In another non-limiting exemplary embodiment, the heating device further comprises a pair of endcaps, wherein one endcap is provided on each operative end of the hollow housing. In one example, the endcaps are made of a resilient material, and may have at least one slit extending thereon for facilitating introduction of the surgical stubes inside the hollow housing.

In another non-limiting exemplary embodiment, a method for heating one or more endotracheal tubes preparatory to nasal intubation, comprises 1) providing one or more endotracheal tubes; 2) providing the heating device 100; 3) receiving the one or more endotracheal tubes through the entrance; 4) heating the one or more endotracheal tubes using the heating device 100; 5) after being heated by the heating device, selecting at least one of the one or more heated endotracheal tubes; and 6) using the selected at least one of the one or more heated endotracheal tubes to perform nasal intubation.

### BRIEF DESCRIPTION OF THE DRAWINGS

FIG. 1 shows a front view of a heating device for surgical tubes, according to an embodiment of the present disclosure.

FIG. 2 shows a perspective view of the heating device device for surgical tubes, according to an embodiment of the present disclosure.

FIG. 3 shows a perspective view of the heating device device depicting the surgical tubes being inserted within the heating device, according to an embodiment of the present disclosure.

FIG. 4 shows perspective views of a hollow housing used in the heating device for the surgical tubes, according to an embodiment of the present disclosure.

FIG. 5 shows perspective view of a heating element being wound on the hollow housing, according to an embodiment of the present disclosure.

FIG. 6 shows a perspective view of the heating element used in the heating device, according to an embodiment of the present disclosure.

FIG. 7 shows a perspective view depicting an insulation layer being assembled over the heating element, according to an embodiment of the present disclosure.

FIG. 8 and FIG. 9 show perspective views depicting endcaps being assembled onto the operative ends of the hollow housing, according to an embodiment of the present disclosure.

FIG. 10 shows a front view of a heating device for surgical tubes, according to another embodiment of the present disclosure.

### DETAILED DESCRIPTION OF EXAMPLE EMBODIMENTS

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

As mentioned previously, surgical tubes such as endotracheal tubes may cause bleeding in the nasal cavity by traumatizing the delicate nasal tissues. In order to overcome this problem, the present disclosure envisages pre-heating the surgical tubes or the endotracheal tubes prior to performing the nasal intubation. Pre-heating the endotracheal tubes prior to the nasal intubation softens the tubes substantially. When a leading edge of the endotracheal tube is softened, insertion thereof inside the nasal cavity is substantially less traumatic for the delicate nasal tissues. Heating the body of the endotracheal tube also makes it easier to pass the tube through the tight and constricted spaces of the nasal passages with significantly less trauma and bleeding.

Another advantageous aspect of pre-heating the endotracheal tube prior to the nasal intubation is that the body of the endotracheal tube may be reshaped to in accordance with a unique individual anatomy, which may then be made set by spraying cold water or saline over the body of the endotracheal tube. An advantageous aspect of having a uniquely formed endotracheal tube is that it reduces the required maneuvering in the form of lateral or longitudinal movement of the tube for successfully performing the nasal intubation.

It is to be noted that the two aforementioned advantageous aspects may be applied by the practitioner simultaneously. More specifically, the leading edge of the endotracheal tube may be heated and softened, and the body of the endotracheal tube may be heated and reshaped to suit the unique individual anatomy of the patient by simply cooling the body of the tube, while keeping the leading edge in its heated softened state.

To this end, the present disclosure envisages a heating device for heating the surgical tubes. In one exemplary application, the surgical tubes may be endotracheal tubes. However, other kinds surgical tubes may be heated as well using the heating device envisaged in the present disclosure. More specifically, the constructional configuration of the heating device may be changed to accommodate different kinds of surgical tubes while using the same concept that is envisaged in the present disclosure.

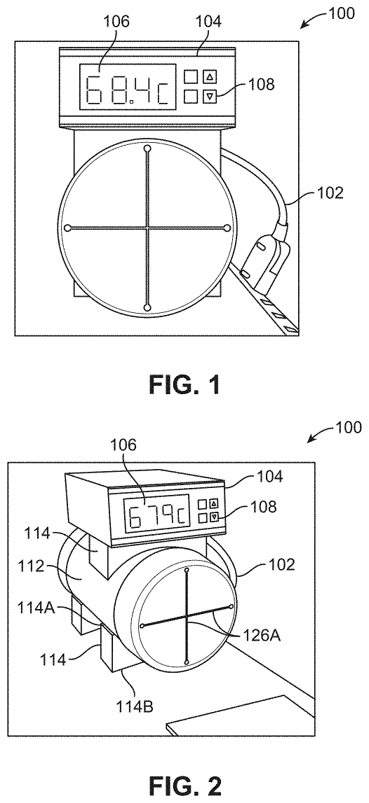

Referring to FIG. 1, a front view of a heating device 100 for surgical tubes, according to an embodiment of the present disclosure, is illustrated. As seen in FIG. 1, the heating device 100 is a small and compact heating device that may be placed tabletop. The heating device 100 has a power cable 102 extending from a controller 104. The power cable 102 may be connected to any suitable power outlet. The controller 104 includes a display 106 and a plurality of buttons 108. In one embodiment, the controller 104 is a microprocessor enabled controlling device. The display 106 and the buttons 108 that allow a practitioner to view, select, and set a desired temperature as a required heating temperature to which the practitioner wishes to heat the surgical tubes. In accordance with one embodiment of the heating device 100, when the heating device 100 turned on via the controller 104, the heating device 100 defaults to the previous temperature setting used by the practitioner. Thus, if a practitioner has a preferred setting, the heating device 100 will operate at that setting when turned on until the practitioner decides to change the required heating temperature setting.

Referring to FIG. 2, a front view of a heating device 100 for surgical tubes, according to an embodiment of the present disclosure, is illustrated. The heating device 100 comprises a hollow housing 112. The hollow housing 112 is that housing in which the surgical tubes to be heated are inserted. In one embodiment, the hollow housing 112 may have a cylindrical configuration, as seen in FIG. 2. However, the configuration of the hollow housing 112 is not restricted to being cylindrical, and the hollow housing 112 may have any other shape, including but not limited to, a rectangular cross section, an elliptical cross section, a polygonal cross section, and the like.

As seen in FIG. 2, the hollow housing 112 is supported on a pair of support brackets 114. The two support brackets 114 are fitted in a spaced apart manner at the operative bottom surface of the hollow housing 112. The brackets 114 define a semi-circular edge 114A that fits onto the hollow housing 112, whereas a bottom edge 114B of the support bracket 114 defines a flat edge that may be placed on a flat support surface, such as that of a tabletop. Identical to the bottom pair of support brackets 114, another support bracket 114 is disposed at an operative top surface of the hollow housing 112 that facilitates the mounting of the controller 104 thereon.

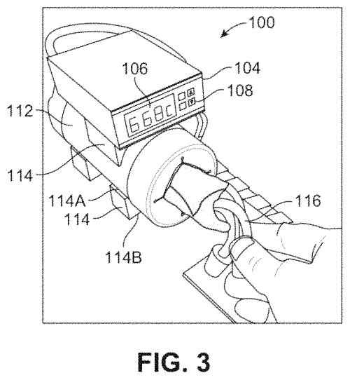

Referring to FIG. 3, a perspective view of the heating device 100 depicting surgical tubes 116 being inserted within the heating device 100, according to an embodiment of the present disclosure, is illustrated. In one exemplary application of the heating device 100, the surgical tubes 116 are endotracheal tubes to be used for nasal intubation. However, the usage of the heating device 100 for heating other kinds of surgical tubes is well within the ambit of the present disclosure.

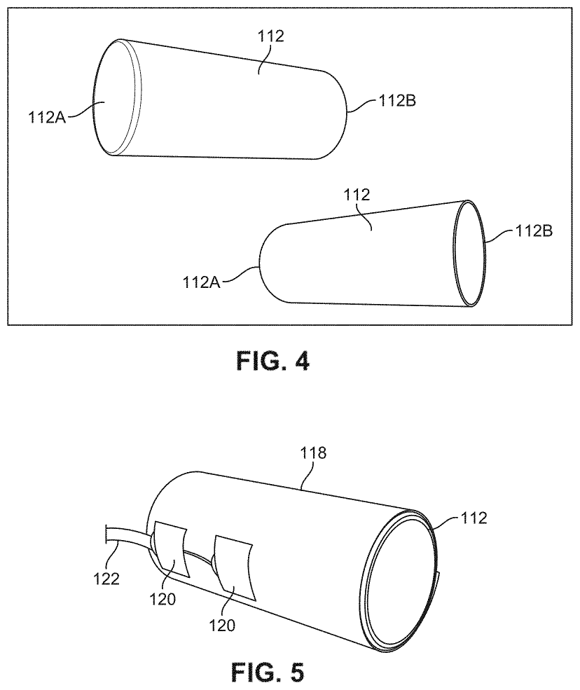

Referring to FIG. 4, perspective views of the hollow housing 112 used in the heating device for the surgical tubes 116, according to an embodiment of the present disclosure, are illustrated. As seen in FIG. 4, the hollow housing 112 is a hollow cylindrical housing or a pipe like structure that is open at both operative ends 112A, 112B thereof. In one embodiment, the hollow housing 112 may be made of a metallic material. In another embodiment, the hollow housing 112 may be made of a non-metallic thermal conductor material, e.g., graphite. It is advantageous if the hollow housing is made of a thermally conductive material because the hollow housing has to receive the heat from a heat source and conduct the same heat into an interior space 112C for facilitating heating of the surgical tubes 116 received inside the interior space 112C.

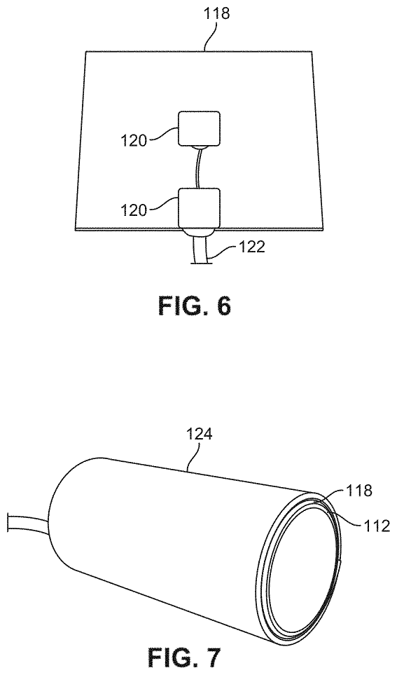

Referring to FIG. 5 and FIG. 6, perspective views of a heating element 118 being wound on the hollow housing 112 and a perspective view of the heating element 118 alone, according to an embodiment of the present disclosure, is illustrated. The heating element 118, in accordance with one exemplary embodiment of the present invention, is a heating pad. The heating element 118 may be wound around the hollow housing 112 and secured thereon by means of an adhesive tape, in accordance with one embodiment of the present disclosure. In one embodiment, the heating element 118 may be secured on the body of the hollow housing 112 by means of a thermally conductive adhesive.

As seen in FIG. 5 and FIG. 6, a pair of first sensors 120 is securely disposed on the heating element 118 in surface contact with the heating element 118 for sensing the temperature of the heating element 118. In accordance with an embodiment of the present disclosure, the pair of first sensors 120 and the heating element 118 are coupled to the controller 104. A cable extends from the hollow housing 112 and terminated into the controller 104. The coupling between the controller 104 and the heating element 118 and between the controller 104 and the pair of first sensors 120 may be facilitated by two different cables that are wrapped together in the form of the cable 122. As mentioned previously, the controller 104 includes the display and buttons provided thereon. The display and the buttons allow the practitioner to view, select, and set the required heating temperature at which the surgical tubes are required to be heated. Once the required heating temperature is set by the practitioner, the controller 104 receives the input regarding the actual temperature of the heating element 118 via the first sensors 120. If the actual temperature has a mismatch with the required heating temperature set by the practitioner on the controller 104, the controller regulates the operation of the heating element 118 accordingly to mitigate the mismatch to zero as soon as possible.

Referring to FIG. 7, a perspective view depicting an insulation layer 124 being assembled over the heating element, according to an embodiment of the present disclosure, is illustrated. The heating device 100 comprises the insultation layer 124 that is assembled over the heating element 118 and the first sensors 120. The insultation layer 124 prevents injury or burns to the practitioner on accidental touch. Another advantageous aspect of the insulation layer 124 is that the insulation layer 124 holds the heating element 118 and the first sensors 120 securely sandwiched between the hollow housing 112 and the insulation layer 124. The insulation layer 124 may be made of a resilient material, in accordance with one embodiment of the present disclosure. The resilient material may be a polymeric material, in accordance with one embodiment.

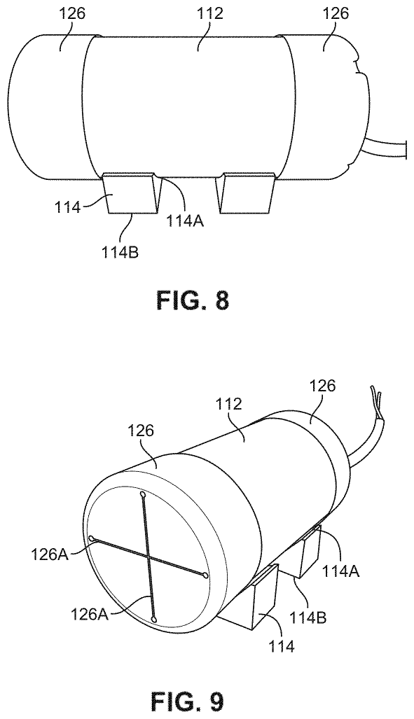

Referring to FIG. 8 and FIG. 9, perspective views depicting endcaps 126 being assembled onto the operative ends 112A, 112B of the hollow housing 112, according to an embodiment of the present disclosure, are illustrated. The endcaps 126, in accordance with an embodiment of the present disclosure, are made of a resilient material, wherein the resilient material may be a polymeric material. The endcaps 126 may include at least one slit 126A configured thereon for allowing the introduction of one or more surgical tubes inside the hollow housing 112. In one embodiment, the endcaps 126 may have two slits 126A, one of which may be configured vertically and other one may be configured horizontally while dissecting the vertical slit to define a cross like configuration. The endcaps 126 with the slits 126A are provided on the ends of the hollow housing 112 to allow introduction of the surgical tubes inside the hollow housing 112 while minimizing the heat loss from the operative ends 112A, 112B of the hollow housing 112 into the surroundings.

In accordance with one aspect of the present disclosure, the practitioner may control the length of the endotracheal tube that is required to be heated simply by controlling how deeply the tubes are inserted into the hollow housing 112. An advantageous aspect of the heating device 100 is that it allows the practitioner to adjust the temperature to meet the required needs for softness of the tubes or to increase or decrease their working time when reshaping the tubes.

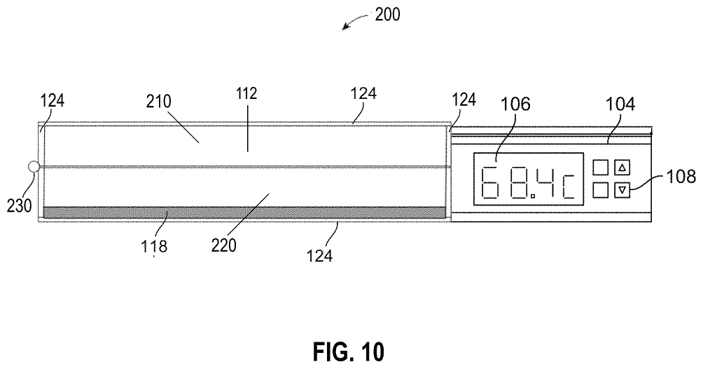

Referring to FIG. 10, a front view of a heating device 200 for surgical tubes, according to another embodiment of the present disclosure, is illustrated.

The heating device 200 is substantially the same as the heating device 100, the main difference being that the hollow housing 112 is shaped as a cuboid (box shaped). More particularly, the housing 112 includes a top portion 210 and a bottom portion 220, and a hinge 230 that pivotally connects the top portion 210 and a bottom portion. In operation, the user opens the heating device 200 by lifting the top portion 220 open and placing the surgical tubes 116 therein. As shown, the insulation layer 124 is disposed around interior surfaces of the heating device 200; however, the insulation layer 124 may be disposed additionally or instead, on exterior surfaces. The heating element 118 and the first sensors 120 can be disposed on the bottom interior surface, as shown, or elsewhere. Although FIG. 10 depicts a box-like structure, it is to be understood that various other shapes and configurations may be used without departing from the spirit and scope of the present invention.

In accordance with an embodiment, a method for heating one or more endotracheal tubes preparatory to nasal intubation, comprises 1) providing one or more endotracheal tubes; 2) providing the heating device 100 or 200; 3) receiving the one or more endotracheal tubes in a hollow portion of the device 100, 200; 4) heating the one or more endotracheal tubes using the heating device 100; 5) after being heated by the heating device, selecting at least one of the one or more heated endotracheal tubes; and 6) using the selected at least one of the one or more heated endotracheal tubes to perform nasal intubation.

Although the features, functions, components, and parts have been described herein in accordance with the teachings of the present disclosure, the scope of coverage of this patent is not limited thereto. On the contrary, this patent covers all embodiments of the teachings of the disclosure that fairly fall within is the scope of permissible equivalents.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A heating device for heating surgical tubes, the heating device comprising:
a hollow housing configured to receive the surgical tubes therein;
a heating element provided in thermal communication with the hollow housing;
a first sensor configured to sense the temperature of the heating element;
a controller coupled to the heating element and the first sensor, wherein the controller is configured to receive an input associated with a required heating temperature from a user for heating the surgical tubes, and accordingly regulate the operation of the heating element based on the required heating temperature and an actual temperature sensed via the first sensor.

2. The heating device according to claim 1, further comprising an insulation layer assembled over the heating element such that the first sensor is sandwiched between the heating element and the insulation layer.

3. The heating device according to claim 1, wherein heating element is a heating pad.

4. The heating device according to claim 2, further comprising a pair of end caps assembled on each operative end of the hollow housing for securely holding the heating element and the insulation layer on the hollow housing.

5. The heating device according to claim 4, wherein the endcaps are made of a resilient material, and the endcaps include at least one slit extending thereon for facilitating introduction of the surgical tubes within the hollow housing.

6. The heating device according to claim 1, wherein the hollow housing is substantially cylindrical.

7. The heating device according to claim 1, wherein the controller includes user interface buttons and a display for allowing the user to view, select, and set an appropriate temperature value as the required heating temperature.

8. The heating device according to claim 1, wherein the hollow housing is substantially cuboid.

9. The heating device according to claim 1, further comprising at least one support bracket for supporting the hollow housing on a flat surface, and for supporting the controller on the hollow housing.

10. The heating device according to claim 1, wherein the surgical tubes include endotracheal tubes.

11. A method for heating one or more endotracheal tubes preparatory to nasal intubation, comprising:
providing the one or more endotracheal tubes;
providing a heating device including:
a hollow housing configured to receive the one or more endotracheal tubes therein through an entrance;
a heating element provided adjacent the hollow housing;
a first sensor configured to sense the temperature of the heating element;
a controller coupled to the heating element and the first sensor, wherein the controller is configured to receive an input associated with a required heating temperature from a user for heating the surgical tubes, and accordingly regulate the operation of the heating element based on the required heating temperature and an actual temperature sensed via the first sensor; and
receiving the one or more endotracheal tubes through the entrance.

12. The method according to claim 11, wherein the heating device further comprises an insulation layer assembled over the heating element such that the first sensor is sandwiched between the heating element and the insulation layer.

13. The method according to claim 11, wherein heating element is a heating pad.

14. The method according to claim 12, wherein the heating device further comprises a pair of end caps assembled on each operative end of the hollow housing for securely holding the heating element and the insulation layer on the hollow housing.

15. The method according to claim 14, wherein the endcaps are made of a resilient material, and the endcaps include at least one slit extending thereon for facilitating introduction of the surgical tubes within the hollow housing.

16. The method according to claim 11, wherein the hollow housing is made of a metallic material.

17. The method according to claim 11, wherein the controller includes user interface buttons and a display for allowing the user to view, select, and set an appropriate temperature value as the required heating temperature.

18. The method according to claim 14, wherein the insulation layer and the endcaps are made of a polymeric material.

19. The method according to claim 11, wherein the heating device further comprises at least one support bracket for supporting the hollow housing on a flat surface, and for supporting the controller on the hollow housing.

20. The method according to claim 11, further comprising:
after being heated by the heating device, selecting at least one of the one or more heated endotracheal tubes; and
using the selected at least one of the one or more heated endotracheal tubes to perform nasal intubation.
